(19) United States
(12) Patent Application Publication
(10) Pub. No.: US 2013/0239965 A1
(43) Pub. Date: Sep. 19, 2013

(54) HUMIDIFICATION SYSTEM WITH SIGNAL TRANSMISSION OPTIMIZATION

(71) Applicants: Benjamin Irwin Shelly, Pittsburgh, PA (US); Bryan Richard McFadden, Derry, PA (US); Mark William Dimatteo, Irwin, PA (US); Mark Barclay, Saxonburg, PA (US); Christopher Scott Lucci, Murrysville, PA (US); Michael Thomas Kane, Harrison City, PA (US)

(72) Inventors: Benjamin Irwin Shelly, Pittsburgh, PA (US); Bryan Richard McFadden, Derry, PA (US); Mark William Dimatteo, Irwin, PA (US); Mark Barclay, Saxonburg, PA (US); Christopher Scott Lucci, Murrysville, PA (US); Michael Thomas Kane, Harrison City, PA (US)

(73) Assignee: KONINKLIJKE PHILIPS ELECTRONICS N.V., EINDHOVEN (NL)

(21) Appl. No.: 13/521,492

(22) PCT Filed: Dec. 17, 2010
(86) PCT No.: PCT/IB10/55919

§ 371 Date: Jul. 11, 2012

Related U.S. Application Data
(60) Provisional application No. 61/295,299, filed on Jan. 15, 2010.

(51) Int. Cl. A61M 16/16; A61M 16/06; A61M 16/08; A61M 16/10; A61M 16/00

(52) U.S. Cl. CPC A61M 16/16; A61M 16/1075; A61M 16/0057; A61M 16/0875; A61M 16/0666
USPC 128/203.26

(57) ABSTRACT

A pressure support device configured to provide pressure support therapy includes a humidifier that holds an enhanced amount of liquid without significantly mitigating sensitivity of respiratory event detection. The humidifier includes a chamber configured to hold liquid, and a partition that divides the chamber such that the volume of the chamber through which a flow of gas generated by the pressure support device flows is reduced. The reduction in the volume of the chamber through which the flow of gas flows preserves respiratory event detection sensitivity.

This patent application claims the priority benefit under 35 U.S.C. §119(e) of U.S. Provisional Application No. 61/295,299 filed on Jan. 15, 2010, the contents of which are herein incorporated by reference.

The invention relates to a pressure support device configured to provide pressure support therapy to a subject, wherein the pressure support device comprises a humidifier configured to control the humidity of gas provided to the subject by the pressure support device.

Pressure support devices that provide pressure support therapy to the airway of a subject are known. Some conventional pressure support devices include humidifiers configured to control the level of humidity of gas provided to the subject during pressure support therapy. In conventional pressure support devices, increasing a capacity to hold liquid for use in the humidifier may enhance the convenience of the pressure support device to users. However, typically, increasing the capacity to hold liquid in a pressure support device humidifier may increase the volume of the flow path formed by the humidifier through which gas must travel on the way to the subject.

One aspect of the invention relates to a pressure support device configured to generate a pressurized flow of breathable gas for delivery to an airway of a subject, the pressure support device comprising a humidifier configured to humidify the pressurized flow of breathable gas. In one embodiment, the humidifier comprises a chamber, a heating element, and a partition. The chamber includes a gas inlet configured to receive a flow of gas into the chamber and a gas outlet configured to release the flow of gas from the chamber. The chamber is configured to hold liquid. The heating element is configured to controllably elevate the temperature of fluid within the chamber to vaporize liquid within the chamber such that the gas flowing through the chamber from the gas inlet to the gas outlet is humidified by the vaporized liquid. The partition is configured to separate the chamber into a first section and a second section. The first section forms a flow path from the gas inlet to the gas outlet. The partition permits fluid communication between liquid held in the first section and liquid held in the second section while restricting contact between the gas within the second section and the pressurized flow of breathable gas as the pressurized flow of breathable gas flows through the first section such that the flow of gas is maintained within the first section.

Another aspect of the invention relates to a method of humidifying a pressurized flow of breathable gas generated by a pressure support device for delivery to an airway of a subject. In one embodiment, the method comprises holding liquid in a chamber that includes a gas inlet configured to receive a flow of gas into the chamber and a gas outlet configured to release the pressurized flow of breathable gas from the chamber; separating a first section of the chamber from a second section of the chamber such that the first section forms a flow path from the gas inlet to the gas outlet, and such that liquid held in the first section and liquid held in the second section are in fluid communication while restricting contact between the gas within the second section and the pressurized flow of breathable gas as the flow of gas flows through the first section such that the flow of gas is maintained within the first section; and controllably elevating the temperature of fluid within a first section of the chamber that forms a flow path between the gas inlet and the gas outlet such that liquid within the first section on of the chamber is vaporized and humidifies gas flowing through the first section of the chamber from the gas inlet to the gas outlet.

Yet another aspect of the invention relates to a pressure support device configured to generate a pressurized flow of breathable gas for delivery to an airway of a subject, the pressure support device comprising a system configured to humidify the pressurized flow of breathable gas. In one embodiment, the system comprises means for holding liquid that includes a gas inlet configured to receive a flow of gas and a gas outlet configured to release the flow of gas; means for separating the means for holding liquid into a first section and a second section such that the first section forms a flow path from the gas inlet to the gas outlet, and such that liquid held in the first section and liquid held in the second section are in fluid communication while restricting contact between the gas within the second section and the flow of gas as the pressurized flow of breathable gas flows through the first section such that the flow of gas is maintained within the first section; and means for controllably elevating the temperature of the liquid such that liquid in the first section is vaporized and humidifies gas flowing through the flow path.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In one embodiment of the invention, the structural components illustrated herein are drawn to scale. It is to be expressly understood, however, that the drawings are for the purpose of illustration and description only and are not a limitation of the invention. In addition, it should be appreciated that structural features shown or described in any one embodiment herein can be used in other embodiments as well. It is to be expressly understood, however, that the drawings are for the purpose of illustration and description only and are not intended as a definition of the limits of the invention.

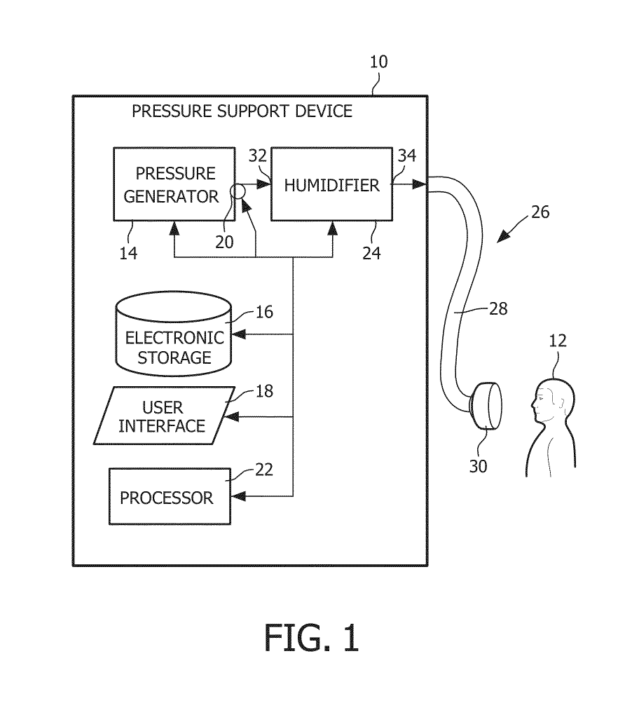

FIG. 1 illustrates a pressure support device configured to provide pressure support therapy to a subject, in accordance with one or more embodiments of the invention;

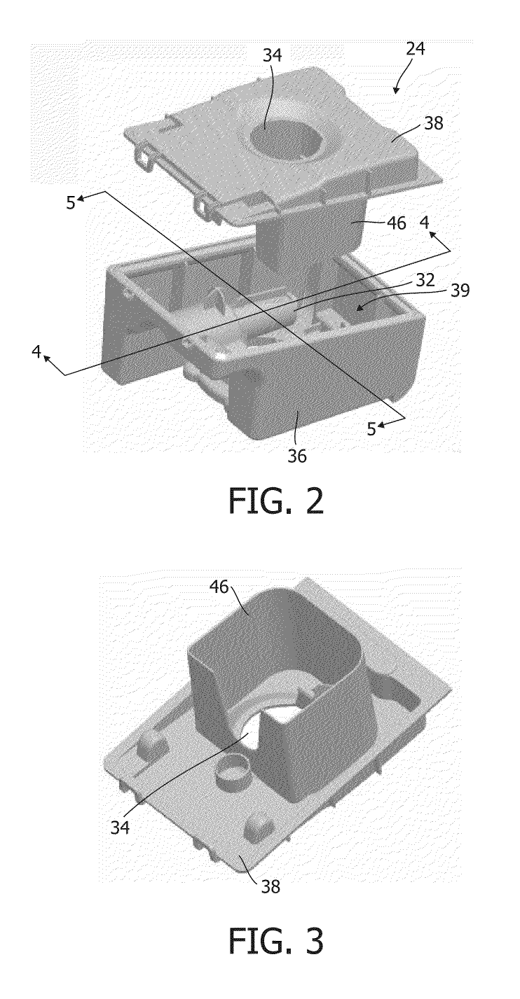

FIG. 2 illustrates a humidifier of a pressure support device, according to one or more embodiments of the invention;

FIG. 3 illustrates a unitary partition structure of a humidifier of a pressure support device, according to one or more embodiments of the invention;

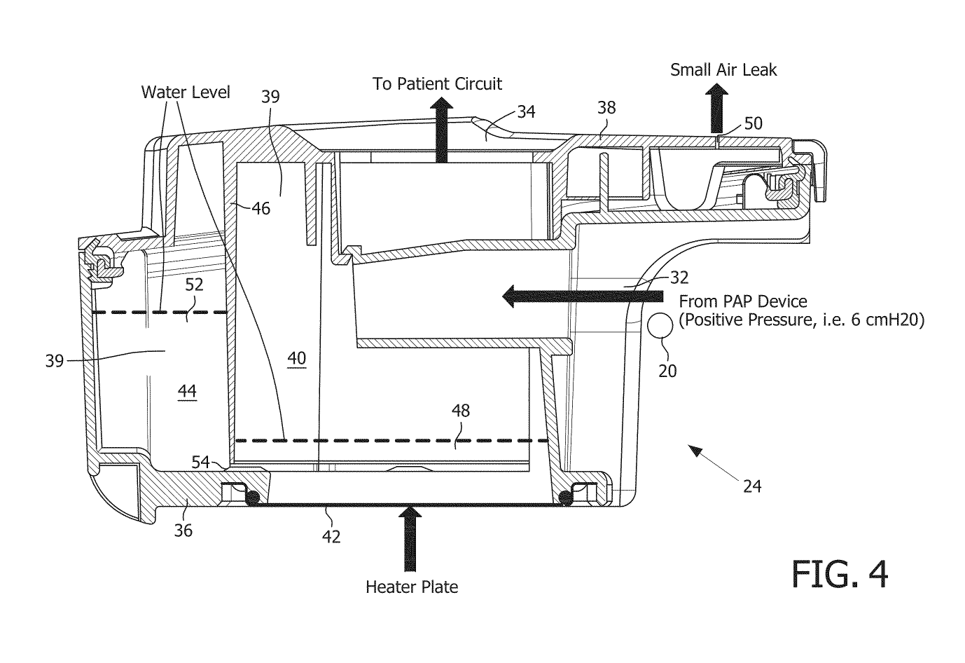

FIG. 4 illustrates a humidifier of a pressure support device, according to one or more embodiments of the invention; and

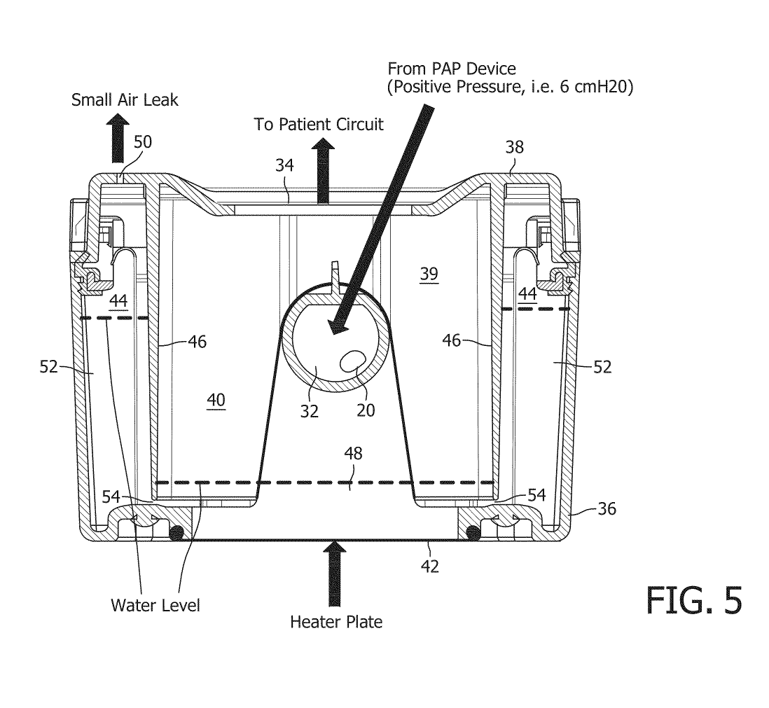

FIG. 5 illustrates a humidifier of a pressure support device, according to one or more embodiments of the invention.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIG. 1 illustrates a pressure support device 10 configured to provide pressure support therapy to a subject 12. Pressure support device 10 is configured to provide the pressure support therapy in the form of a pressurized flow of breathable gas that is delivered to the airway of subject 12. The pressure support therapy may be dynamic in that one or more parameters of the pressurized flow of breathable gas generated by pressure support device 10 may be adjusted based on detection of one or more parameters. For example, the pressure of the pressurized flow of breathable gas may be increased based on changes to one or more parameters that indicate a respiratory event (e.g., an apnea, snoring, etc.). In one embodiment, pressure support device 10 includes one or more of a pressure generator 14, electronic storage 16, a user interface 18, a sensor 20, a processor 22, a humidifier 24, and/or other components.

In one embodiment, pressure generator 14 is configured to generate a pressurized flow of breathable gas for delivery to the airway of subject 12. Pressure generator 14 may control one or more parameters of the pressurized flow of breathable gas (e.g., flow rate, pressure, volume, humidity, temperature, gas composition, etc.) for therapeutic purposes, or for other purposes. By way of non-limiting example, pressure generator 14 may be configured to control the flow rate and/or pressure of the pressurized flow of breathable gas to provide pressure support to the airway of subject 12. The pressure generator may include a ventilator, a positive airway pressure generator such as, for example, the pressure generator of the device described in U.S. Pat. No. 6,105,575, hereby incorporated by reference in its entirety, and/or other pressure generation devices.

The pressurized flow of breathable gas is delivered to the airway of subject 12 from pressure support device 10 via a gas delivery circuit 26. Gas delivery circuit 26 is configured to communicate the pressurized flow of breathable gas generated by pressure generator 14 to the airway of subject 12. As such, gas delivery circuit 26 includes a conduit 28 and an interface appliance 30. Conduit 28 conveys the pressurized flow of breathable gas to interface appliance 30, and the interface appliance delivers the pressurized flow of breathable gas to the airway of subject 12. Some examples of interface appliance 30 may include, for example, a nasal cannula, a nasal mask, a nasal/oral mask, a full face mask, a total face mask, and/or other interface appliances that communication a flow of gas with an airway of a subject. The present invention is not limited to these examples, and contemplates delivery of the pressurized flow of breathable gas to subject 12 using any subject interface.

Although gas delivery circuit 26 is illustrated in FIG. 1 as a single-limbed circuit for the delivery of the pressurized flow of breathable gas to the airway of subject 12, this is not intended to be limiting. The scope of this disclosure includes double-limbed circuits having a first limb configured to both provide the pressurized flow of breathable gas to the airway of subject 12, and a second limb configured to selectively exhaust gas from gas delivery circuit 26 (e.g., to exhaust exhaled gases).

In one embodiment, electronic storage 16 comprises electronic storage media that electronically stores information. The electronic storage media of electronic storage 16 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 16 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 16 may store software algorithms, information determined by processor 22, information received via user interface 18, and/or other information that enables system 10 to function properly. Electronic storage 16 may be (in whole or in part) a separate component within system 10, or electronic storage 16 may be provided (in whole or in part) integrally with one or more other components of system 10 (e.g., generator 14, user interface 18, processor 22, etc.).

User interface 18 is configured to provide an interface between system 10 and subject 12 through which subject 12 may provide information to and receive information from system 10. This enables data, cues, results, and/or instructions and any other communicable items, collectively referred to as “information,” to be communicated between the subject 12 and one or more of generator 14, electronic storage 16, and/or processor 22. Examples of interface devices suitable for inclusion in user interface 18 include a keypad, buttons, switches, a keyboard, knobs, levers, a display screen, a touch screen, speakers, a microphone, an indicator light, an audible alarm, a printer, a tactile feedback device, and/or other interface devices. In one embodiment, user interface 18 includes a plurality of separate interfaces. In one embodiment, user interface 18 includes at least one interface that is provided integrally with generator 14.

It is to be understood that other communication techniques, either hard-wired or wireless, are also contemplated by the present invention as user interface 18. For example, the present invention contemplates that user interface 18 may be integrated with a removable storage interface provided by electronic storage 16. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 18 include, but are not limited to, an RS-232 port, RF link, an IR link, modem (telephone, cable or other). In short, any technique for communicating information with system 10 is contemplated by the present invention as user interface 18.

Sensor 20 is configured to generate output signals conveying information related to one or more parameters of the pressurized flow of breathable gas and/or the breathing of subject 12. The one or more parameters of the pressurized flow of breathable gas may include, for example, one or more of a flow rate, a volume, a pressure, humidity, temperature, acceleration, velocity, acoustics, changes in a parameter indicative of respiration, and/or other gas parameters. Sensor 20 may include one or more sensors that measure such parameters directly (e.g., through fluid communication with the pressurized flow of breathable gas at pressure generator 14). The sensor 20 may include one or more sensors that generate output signals related to one or more parameters of the pressurized flow of breathable gas indirectly. For example, sensor 20 may include one or more sensors configured to generate an output based on an operating parameter of pressure generator 14 (e.g., a valve driver or motor current, voltage, rotational velocity, and/or other operating parameters), and/or other sensors. The one or more parameters of the breathing of the subject (that are not parameters of the pressurized flow of breathable gas) may include other parameters that provide information about the breathing of subject 12. For example, sensor 20 may include a transducer configured to detect acoustic waves transmitted to pressure support device 10 through gas delivery circuit 26. These acoustic waves may convey information related to respiratory effort of subject 12, and/or the noise generated by subject 12 during respiration (e.g., during snoring).

Although sensor 20 is illustrated as a single sensor at a single location in pressure generator 14, this is not intended to be limiting. The sensor 20 may include a plurality of sensors which may be located proximately or separately with respect to each other. Sensors providing the functionality attributed herein to sensor 20 may be disposed in any of a plurality of locations, such as for example, within pressure generator 14, within (or in communication with) conduit 28, within (or in communication with) interface appliance 30, and/or other locations.

Processor 22 is configured to provide information processing capabilities in system 10. As such, processor 22 may include one or more of a digital processor, an analog processor, a digital circuit designed to process information, an analog circuit designed to process information, a state machine, and/or other mechanisms for electronically processing information. Although processor 22 is shown in FIG. 1 as a single entity, this is for illustrative purposes only. In some implementations, processor 22 may include a plurality of processing units. These processing units may be physically located within the same device (e.g., pressure generator 14), or processor 22 may represent processing functionality of a plurality of devices operating in coordination.

Processor 22 is configured to control pressure generator 14 to generate the pressurized flow of breathable gas in accordance with the therapy regime. By way of non-limiting example, processor 22 may control pressure generator 14 such that the pressure support provided to subject 12 via the pressurized flow of breathable gas includes, non-invasive ventilation, positive airway pressure support, bi-level support, continuous positive airway pressure support, BiPAP®, and/or other types of pressure support therapy.

In controlling pressure generator 14, the therapy regime may dictate that processor 22 be responsive to the output signals generated by sensor 20. For example, if the output signals generated by sensor 20 indicate that subject 12 is experiencing a respiratory event, the therapy regime may dictate that the processor control pressure generator 14 to increase pressure of the pressurized flow of breathable gas to help subject 12 overcome the event. Some non-limiting examples of respiratory events include an apnea (central or obstructive), a respiratory obstruction, snoring, hypopnea, flow limitation, and/or other respiratory events.

Humidifier 24 is configured to adjust the humidity of the pressurized flow of breathable gas. In one embodiment, humidifier 24 is configured to generate water vapor by heating liquid held within the humidifier. Humidifier 24 includes a gas inlet 32 and a gas outlet 34. Humidifier 24 is configured such that the pressurized flow of breathable gas is received from pressure generator 14 by humidifier 24 through gas inlet 32 and is humidified within humidifier 24 by water vapor before being released from humidifier 24 through gas outlet 34. In one embodiment, gas outlet 34 is connected with gas delivery circuit 26 such that the humidified pressurized flow of breathable gas is delivered to the airway of subject 12 through gas delivery circuit 26.

Humidifier 24 is configured such that the amount by which the humidity of the pressurized flow of breathable gas is adjusted within humidifier 24 is controlled by processor 22. For example, processor 22 may control a heating element (not shown in FIG. 1) configured to heat liquid within humidifier 24 to adjust the amount of moisture added to the pressurized flow of breathable gas within humidifier 24. The level of humidity to which the pressurized flow of breathable gas is adjusted may be dictated by a therapy regime and/or selected by a user (e.g., subject 12, a caregiver, a therapy decision-maker, etc.).

In one embodiment, sensor 20 includes one or more sensors generating output signals implemented by processor 22 to detect respiratory events are disposed on a side of humidifier 24 opposite from gas delivery circuit 26. For example, the one or more sensors may be located within pressure generator 14.

In conventional pressure support devices, the sensitivity of such sensors (e.g., sensors disposed on an opposite side of a humidifier from a subject) to respiratory events may be impacted by the size of the humidifier and/or the airspace in the humidifier. Typically, the more liquid that is held by a humidifier of a pressure support device, the greater the volume of a liquid storage chamber (to accommodate the increased amount of liquid) through which gas and/or acoustic waves must pass before coming into contact with the event detecting sensors. Similarly, to reduce occurrences of water ingress (e.g., at startup, during transport of pressure support device 10, and/or at other times) the volume of a chamber that holds water may be increased. Unfortunately, increasing the volume of this storage chamber tends to attenuate the parameters detected by the event detecting sensors. For example, periodic pressure modulation and/or acoustic waves associated with some respiratory events (e.g., apneas, obstructions, snoring, etc.) are typically attenuated more by larger storage chambers.

As such, in conventional designs, there is a design conflict between the amount of liquid that can be held by the humidifier (i.e., the size and configuration of the humidification system) and the sensitivity of respiratory event detection. While holding a larger amount of liquid may enhance the convenience of the pressure support device, reducing the sensitivity of respiratory event detection may reduce the efficacy and/or comfort of the pressure support therapy delivered by the pressure support device. As will be discussed further below. Humidifier 24 of pressure support device 10 is designed to hold an enhanced amount of liquid while maintaining respiratory event detection sensitivity.

FIG. 2 illustrates an exploded view of one or more implementations of humidifier 24. In the view shown in FIG. 2, a unitary base structure 36 is exploded from a unitary partition structure 38. The orientation of unitary base structure 36 and unitary partition structure 38 shown is the orientation in which humidifier 24 would be deployed during use (e.g., with unitary base structure 36 positioned underneath unitary partition structure 38). The unitary base structure 36 and/or unitary partition structure 38 may be formed from polycarbonate, plastic, and/or other materials. Unitary base structure 36 forms gas inlet 32, and unitary partition structure 38 forms gas outlet 34. As is discussed below, a flow path is formed between gas inlet 32 and gas outlet 34 by unitary base structure 36 and unitary partition structure 38. FIG. 3 illustrates reverse elevation of one or more implementations of unitary partition structure 38. It will be appreciated that the description of humidifier 24 as being formed by unitary base structure 36 and unitary partition structure 38 is not intended to be limiting. The scope of this disclosure includes apparatuses having more or fewer pieces, and/or with pieces having different specific shapes.

FIGS. 4 and 5 illustrate sectional views of humidifier 24 with unitary base structure 36 and unitary partition structure 38 assembled. Specifically, FIG. 4 shows a sectional view taken along section line 4-4, and FIG. 5 shows a sectional view taken along section line 5-5. When assembled, humidifier 24 includes a chamber 39, a heating clement 42, and a partition 46.

Chamber 39 is configured to hold liquid, and forms a flow path from gas inlet 32 to gas outlet 34. As such, the pressurized flow of breathable gas flowing through humidifier 24 flows through the flow path defined by chamber 39 between gas inlet 32 and gas outlet 34. During operation, the temperature of at least a portion of the liquid held in chamber 39 is elevated to vaporize the liquid. The pressurized flow of breathable gas is humidified by the vaporized liquid as it flows through the flow path. The base of chamber 39 is formed by unitary base structure 36. The chamber is divided into a first section 40 and a second section 44 by partition 46.

The flow path formed by chamber 39 is formed entirely within first section 40. A ceiling of first section 40 is formed by unitary partition structure 38. First section 40 holds a first reservoir of liquid 48. During use, the portion of the liquid held by chamber 39 that is vaporized is the first reservoir of liquid 48. The first section is sealed (or substantially sealed) from ambient atmosphere).

Heating element 42 is configured to controllably elevate the temperature of fluid within first section 40. In the embodiment shown in FIGS. 4 and 5, heating element 42 is positioned at the bottom of first section 40 to be in proximity to first reservoir of liquid 48 such that heat emitted by heating element 42 is dispensed directly into first reservoir of liquid 48. This emission of heat by heating element 42 into first reservoir of liquid 48 vaporizes first reservoir of liquid 48. As was discussed above with respect to FIG. 1, the amount of heat emitted by heating element 42 is controllable by a processor (e.g., processor 22 shown in FIG. 1 and described above) to bring the humidity of the pressurized flow of breathable gas to a selected level. As can be seen in FIGS. 4 and 5, in one embodiment, heating element 42 does not directly heat fluid within second section 44.

Second section 44 is positioned adjacent to first section 40. In one embodiment, second section 44 at least partially surrounds first section 40. For example, in FIGS. 4 and 5, second section 44 surrounds first section 40 on three sides. An outer side wall of first section 40 is formed by unitary base structure 36. In one embodiment, second section 44 is held in isolation from ambient atmosphere. In one embodiment, a secondary opening 50 in humidifier 24 provides for restrictive communication between second section 44 and ambient atmosphere. Second section 44 is configured to hold a second reservoir of liquid 52.

As was mentioned above, partition 46 is configured to divide first section 40 from second section 44. In dividing first section 40 from second section 44, partition 46 defines an opening 54 between first section 40 and second section 44. Opening 54 is located such that first reservoir of liquid 48 is placed in fluid communication with second reservoir of liquid 52. For example, in the implementations shown in FIGS. 4 and 5, opening 54 is formed toward the bottom of first section 40 and second section 44. More specifically, partition 46 is formed to extend not all the way to the base of first section 40 and second section 44 such that opening 54 is formed underneath partition 46. The gap between the bottom of partition 46 and unitary base structure 36 forming opening 54 may be about 1.5 mm. In one embodiment, partition 46 and opening 54 are formed such that fluid communication between first reservoir of liquid 48 and second reservoir of liquid 52 is unrestricted by any valve or nozzle, but instead enables liquid to pass back and forth between first reservoir of liquid 48 and second reservoir of liquid 52.

The size and/or shape of opening 54 enables gas within first section 40 to be held in isolation from gas within second section 44 even as first reservoir of liquid 48 and second reservoir of liquid 52 are in fluid communication. For example, as is shown in FIGS. 4 and 5, opening 54 may be formed toward the bottom of chamber 39 such that until the amount of liquid held in chamber 39 falls to about 5% of capacity, or some other level near empty, the gas held in first section 40 is isolated from gas in second section 44.

Because the flow path between gas inlet 32 and gas outlet 34 is formed wholly within first section 40, and because this flow path is isolated from second section 44, the volume of chamber 39 through which the pressurized flow of breathable gas passes is effectively reduced to just first section 40. This reduction in the volume of chamber 39 through which the pressurized flow of breathable gas passes reduces the attenuation of parameters that are detected by sensor 20 within a pressure generator (or between humidifier 24 and the pressure generator) to facilitate respiratory event detection. Because second reservoir of liquid 52 is still in fluid communication with first reservoir of liquid 48, this reduction in the attenuation of respiratory event detection sensitivity by sensor 20 is achieved while still providing the advantages of increased liquid storage capacity within humidifier 24.

In one embodiment, opening 54 is formed such that the cross-sectional shape and/or area of opening 54, in conjunction with typically submersed location of opening 54, maintains first section 40 substantially in acoustic isolation from second section 44. The substantial acoustic isolation of first section 40 from second section 44 while still permitting fluid communication between first reservoir of liquid 48 and second reservoir of liquid 52 maintains respiratory event detection sensitivity by sensor 20, but provides for the advantages associated with increased liquid storage within humidifier 24 and/or increased volume within chamber 39 at the same time.

During operation, the pressure within first section 40 is increased by the pressure of the pressurized flow of breathable gas as it flows through first section 40. This increase in pressure is substantial, and may increase the pressure in first section 40 to at least about 4 cmH₂O. Opening 54 is formed such that in response to this increase in pressure in first section 40, liquid in the first reservoir of liquid 48 can flow from first section 40 through opening 54 and into second section 44 to join second reservoir of liquid 52. Because opening 54 does not enable gas within second section 44 to communicate directly with gas in first section 40, the flow of liquid from the first reservoir of liquid 48 to the second reservoir of liquid 52 causes the level of liquid in the second reservoir of liquid 52 to rise and the level of liquid in the first reservoir of liquid 48 to rise. For example, FIGS. 4 and 5 illustrate the manner in which the liquid levels in first section 40 and second section 44 may be adjusted by pressurization of first section 40 by the pressurized flow of breathable gas.

It will be appreciated, however, that the changes in the levels of first reservoir of liquid 48 and second reservoir of liquid 52 caused by increased pressure in first section 40 does not impede replenishment of first reservoir of liquid 48 from second reservoir of liquid 52. As liquid from first reservoir of liquid 48 is vaporized and carried out of first section 40 by the pressurized flow of breathable gas, liquid from second reservoir of liquid 52 passes through opening 54 to replenish first reservoir of liquid 48.

In one embodiment, rather than permitting the pressurization of first section 40 to cause the disparity in liquid levels between the first reservoir of liquid 48 and the second reservoir of liquid 52, one or more openings (not shown) are formed in partition 46 to provide for restrictive communication between the gas held in second section 44 and first section 42. However, the openings are restrictive enough that partition 46 still restricts contact between the gas within the second section 44 and the pressurized flow of breathable gas as the pressurized flow of breathable gas flows through the first section 40 such that the pressurized flow of breathable gas is maintained within the first section. This means that the openings restrict communication between the first section 40 and the second section 44 to the point that the flow path through which the pressurized flow of breathable gas travels does not include the second section 44. Thus, sudden changes in pressure and/or flow rate experienced by the pressurized flow of breathable gas due to, for example, respiration by subject 12 are not dampened by the volume of the second section 44. For example, in one embodiment, the opening(s) in partition 46 are formed to restrict contact of the pressurized flow of breathable gas with the gas in the second section to a flow rate of less than about 2 LPM at a pressure of about 10 cmH₂O, less than about 3 LPM at a pressure of about 10 cmH₂O, less than about 4 LPM at a pressure of about 10 cmH₂O.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A humidifier configured for use in a pressure support system, the humidifier comprising:
a chamber configured to carry liquid, the chamber comprising a gas inlet and a gas outlet (34), the gas inlet being configured to receive a pressurized flow of breathable gas into the chamber, and the gas outlet being configured to release the pressurized flow of breathable gas from the chamber;
a partition configured to separate the chamber into a first section and a second section, the first section forming a flow path from the gas inlet to the gas outlet for the pressurized flow of breathable gas to pass through the chamber, the partition permitting fluid communication between liquid held in the first section and liquid held in the second section, the partition maintaining the pressurized flow of breathable gas within the first section by at least partially restricting contact between gas within the second section and the pressurized flow of breathable gas as the pressurized flow of breathable gas passes through the first section; and
a heating element configured to controllably elevate a temperature of liquid within the chamber to vaporize at least a portion of the liquid within the chamber such that the pressurized flow of breathable gas passing through the first section of the chamber from the gas inlet to the gas outlet is humidified by the vaporized liquid;
wherein a base surface of the first section and the second section of the chamber, and at least one side wall of the second section of the chamber are formed by a unitary base structure; and
wherein the partition and a ceiling of the first section of the chamber are formed by a unitary partition structure.

2. The humidifier of claim 1, wherein the second section of the chamber at least partially surrounds the first section of the chamber.

3. The humidifier of claim 1, wherein the gas within the second section of the chamber is in restrictive communication with the first section of the chamber such that gas is exchanged between the first section of the chamber and the second section of the chamber at less than about 2 LPM at about 10 cmH₂O.

4. The humidifier of claim 1, wherein the partition is configured such that fluid communication between liquid in the first section and the second section enables liquid held by the second section to replenish the liquid within the first section as the liquid within the first section is vaporized and carried out of the chamber by the pressurized flow of breathable gas.

5. (canceled)

6. A method of humidifying a flow of gas generated by a pressure support device for delivery to an airway of a subject, the method comprising:
holding liquid in a chamber that includes a gas inlet configured to receive a pressurized flow of breathable gas into the chamber and a gas outlet configured to release the pressurized flow of breathable gas from the chamber;
separating a first section of the chamber from a second section of the chamber such that the first section forms a flow path from the gas inlet to the gas outlet, and such that liquid held in the first section and liquid held in the second section are in fluid communication, while at least partially restricting contact between the gas within the second section and the pressurized flow of breathable gas as the pressurized flow of breathable gas passes through the first section such that the pressurized flow of breathable gas is maintained within the first section; and
controllably elevating the temperature of fluid within the first section of the chamber that forms a flow path between the gas inlet and the gas outlet such that liquid within the first section of the chamber is vaporized and humidifies the pressurized flow of breathable gas passing through the first section of the chamber from the gas inlet to the gas outlet;
wherein a base surface of the chamber, and at least one side wall of the second section of the chamber are formed by a unitary base structure; and
wherein the first section of the chamber and the second section of the chamber are separated by a unitary partition structure that also forms a ceiling of the first section of the chamber.

7. The method of claim 6, wherein the second section of the chamber at least partially surrounds the first section of the chamber.

8. The method of claim 6, wherein gas within the second section of the chamber is in restrictive communication with the first section of the chamber such that gas is exchanged between the first section of the chamber and the second section of the chamber at less than about 2 LPM at about 10 cmH₂O.

9. The method of claim 6, further comprising replenishing the liquid within the first section of the chamber from the second section of the chamber as the liquid within the first section of the chamber is vaporized and carried out of the chamber by the pressurized flow of breathable gas.

10. (canceled)

11. A humidification system for use in a pressure support system configured to generate a pressurized flow of breathable gas for delivery to an airway of a subject, the humidification system comprising:
means for holding liquid, including a gas inlet configured to receive a pressurized flow of breathable gas and a gas outlet configured to release the pressurized flow of breathable gas;
means for separating the means for holding liquid into a first section and a second section such that the first section forms a flow path from the gas inlet to the gas outlet, and such that liquid held in the first section and liquid held in the second section are in fluid communication while at least partially restricting contact between the gas within the second section and the pressurized flow of breathable gas as the pressurized flow of breathable gas flows through the first section such that the pressurized flow of breathable gas is maintained within the first section; and
means for controllably elevating the temperature of the liquid such that liquid in the first section is vaporized and humidifies the pressurized flow of breathable gas passing through the flow path;
wherein a base surface and at least one side wall of the means for holding liquid are formed by a unitary base structure; and
wherein the means for separating and a ceiling of the means for holding liquid are formed by a unitary partition structure.

12. The humidification system of claim 11, wherein the second section of the means for holding liquid at least partially surrounds the first section of the means for holding liquid.

13. The humidification system of claim 11, wherein gas within the second section of the chamber is in restrictive communication with the first section of the chamber such that gas is exchanged between the first section of the chamber and the second section of the chamber at less than about 2 LPM at about 10 cmH₂O.

14. The humidification system of claim 11, wherein the means for dividing is configured such that liquid held by the second section of the means for holding liquid replenishes the liquid held by the first section of the means for holding liquid as the liquid within the first section of the means for holding liquid is vaporized and carried out of the means for holding liquid by the pressurized flow of breathable gas passing through the flow path.

15. (canceled)
